(19) United States
(12) Patent Application Publication
(10) Pub. No.: US 2021/0037813 A1
(43) Pub. Date: Feb. 11, 2021

(54) Techniques for Handling Human Organ During Transport

(71) Applicant: UNIVERSITY OF MARYLAND, BALTIMORE, Baltimore, MD (US)

(72) Inventors: Joseph R. Scalea, Baltimore, MD (US); Stephen Restaino, Columbia, MD (US)

(21) Appl. No.: 16/963,446

(22) PCT Filed: Jan. 18, 2019
(86) PCT No.: PCT/US19/14124

§ 371 Date: Jul. 20, 2020

Related U.S. Application Data
(60) Provisional application No. 62/664,352, filed on Apr. 30, 2018.
(60) Provisional application No. 62/619,337, filed on Jan. 19, 2018.

(51) Int. Cl. A01N 1/02; G08C 17/02; G16H 10/60

(52) U.S. Cl. CPC A01N 1/0273; G16H 10/60; G08C 17/02

(57) ABSTRACT

Techniques are provided for the monitoring and transport of an anatomical organ with a sleeve arranged snuggly around the organ and comprising a porous material with respect to an aqueous solution in a container affixed to unmanned aerial vehicle. As a non-limiting example, in a first set of embodiments, the sleeve, shaped to an approximate shape of the target anatomical organ has an opening for inserting the target anatomical organ. The sleeve may comprise a fabric that is porous with respect to an aqueous solution and has a tensile strength to hold the weight of the sleeve and of the target anatomical organ. In other embodiments, the fabric of the sleeve may also include a different second opening for passing a blood vessel for the target anatomical organ. Further, the sleeve may include a wireless communication device and a at least one of a temperature sensor and vibration sensor.

### CROSS-REFERENCE TO RELATED APPLICATIONS

This application claims benefit of U.S. Provisional Appln. 62/619,337, filed Jan. 19, 2018, and of U.S. Provisional Appln. 62/664,352, filed Apr. 30, 2018, the entire contents of each of which are hereby incorporated by reference as if fully set forth herein, under 35 U.S.C. § 119(e).

### BACKGROUND

There is a woeful disparity between the number of Americans on the transplant waiting list and the number of organs available for transplantation. Indeed, in the United States as many as 20 people die every day waiting for a life-saving organ. To take advantage of every organ available, patients, doctors, and other healthcare professionals in the United States utilize the Organ Procurement and Transplantation Network (OPTN)—organized and administered by the United Network of Organ Sharing (UNOS).

UNOS is a non-profit organization located in Richmond, Va. with the purpose of aiding and facilitating the organ transplant and donation process. In addition to managing the national transplant waiting list, maintaining databases on all the transplant events that occur in the U.S., and providing assistance to patients and family members; UNOS also develops policy and procedures for the transplant process.

An essential aspect of improving the organ transplantation process is streamlining and updating the transportation, storage, and monitoring of the organ. Kidneys are the most commonly transplanted organ in the United States, however there are far too few kidneys to meet the needs of those on the transplant waiting list. Extensive work has been done to determine the best utilization of what kidneys are available for transplantation. Culminating in 2014, 12 years of research by transplant professionals showed that too many patients were dying on the kidney transplant waiting list.

In addition, there were disparities in access to transplantation for various patient groups, particularly those patients with a poor immunological profile. As such, complex mathematical models showed that increased organ sharing at a national level could partly remedy this problem. Accordingly, UNOS changed the allocation system—for kidneys specifically—and these changes went into effect on Dec. 4, 2014. The changes allowed for a better donor-recipient immunologic match, thereby increasing the number of potential transplants. After the KAS was updated, sharing organs nationally became more common. After the adoption of the new kidney allocation system, the percentage of kidneys shared nationally (as opposed to remaining local) increased by more than 40% (see Table 1). Indeed, whereas only 20% of kidneys were shared prior to the update, now more than 33% of organs are shared between organ procurement organizations (OPOs).

While the new allocation system demonstrated improved access to transplantation, the distances between the donor and the recipient also increased. For instance, the distance traveled by a typical kidney being transplanted increased, on average, from 197 miles to 267 miles; in some cases, the travel distance increased from 440 miles to 706 miles—a 60% increase in mileage. The longer distances naturally led to longer transit times. In medical terms, the time that an organ spends between the chilling of the organ after its blood supply has been reduced or cutoff and the time it is warmed by having its blood supply restored is referred to as cold ischemia time (CIT).

CIT is a notable predictor of long-term patient and kidney survival. Increased CIT contributes to a problem called delayed graft function (DGF) wherein a kidney may not work immediately after transplantation. The mean CIT has risen such that more than 22% of kidneys are now transplanted after more than 24 hours. This is significant because 24 hours is the accepted “upper limit” for CIT. Accordingly, the rate of DGF among kidney recipients has also increased from 25 to 31% leading to a corresponding number of kidneys which fail to work immediately. Although DGF is treatable, the treatment is exorbitantly expensive, adding, depending on the degree of DGF, as much as $100,000-$250,000 to each transplant procedure (total national cost in excess of $800M per year). More efficient methods for transporting organs could not only improve access to transplantation but reverse the trends in CIT and DGF to pre-KAS levels.

TABLE 1
Geographic Sharing of Organs Based on Changes to
the Kidney Allocation System (KAS) in the USA

| | Before KAS | | After KAS | | | |
|---|---|---|---|---|---|---|
| | N | % | N | % | % change | p-value |
| Local | 8569 | 78.6 | 7805 | 68.5 | −12.90% | <0.0001 |
| Regional | 961 | 8.8 | 1450 | 12.7 | 44.3 | <0.0001 |
| National | 1371 | 12.6 | 2141 | 18.8 | 49.4 | <0.0001 |

Another significant factor in the transportation of organs is the risk to organ transplant personnel. In a recent study of more than 2,000 abdominal and thoracic organ transplant procurements, investigators observed that recovery teams typically travelled between 550-1,066 person-miles to obtain organs for transplantation. Further, it is well established that travel is associated with increased accident risk. Indeed, a recent tragedy in Michigan left 6 dead (4 medical team members and 2 pilots) while en route to procure a life-saving lung transplant. The Michigan team had been traveling in a small 8-seat fixed wing aircraft when they crashed. Even prior to the newly introduced allocation system—and associated increase in CIT—the distance traveled by a typical kidney was about 200 miles, thus requiring significant travel for the transport teams. Given the risk of automobile collisions and high-risk travel, typically at night, for surgical staff on light-aircraft (often in small fixed-wing propeller aircraft and helicopters), removing these travel risks to both pilots and surgical recovery staff remains an important, yet unmet need in the field of transplantation.

While many kidneys are transported by commercial travel, it is an inadequate solution. It is frequently the case that commercial aviation schedules and travel times are inconsistent with optimal organ delivery times. As such, the use of commercial flights does not adequately meet the needs of transplant professionals. It is the undersigned's experience that life-saving organs are often declined for transplantation because, as a result of the commercial airline schedule, the target organs will incur markedly prolonged CITs.

Additionally, current costs of organ procurement are staggeringly high. Recently, the undersigned inventors were part of a transplantation procedure with a donor liver originating in Texas; for the donor liver to arrive at the transplantation center in Maryland, a private jet charter was required at a charge of $80,000. In a similar situation, the transplantation center in Maryland incurred a transportation charge of nearly $30,000 for a chartered jet flight to procure a life-saving combined kidney-pancreas transplant. The average cost of a transplantable kidney is approximately $40,000, and a substantial percentage of this cost is transportation charge. As such, the United States spends more than $680M annually on transplantable kidneys.

Moreover, during transportation and manipulation, organs are subjected to a host of unique environmental conditions and changes that impact the viability and survivability of the organ. For instance, changes in vibration, pressure, and temperature all affect the organ tissue and are determinative on whether the organ is suitable for transplantation once it arrives at its destination. Moreover, these conditions and changes often go undetected by the transplant personnel as there is little to no record of the conditions to which the organ was exposed.

### SUMMARY

Significant improvements in lessening CITs could be achieved using unmanned aerial systems (UAS) for organ transportation thereby improving the availability of transplantable organs. Potential improvements in organ quality—resulting from expedited travel—could improve organ utilization, reduce the number of discarded organs, increase the number of transplantable marginal organs which might otherwise be discarded, and improve transplant outcome of organ transplantation. UAS organ transportation represents a potentially enormous opportunity in the world of transplantation. Techniques are provided to enable the utilization of UAS and other advancements in organ transport by monitoring unique environmental conditions and changes that impact the viability and survivability of the organ.

Techniques are provided for the monitoring and transport of an anatomical organ. These techniques include utilizing a sleeve arranged snuggly around the organ and comprising a porous material with respect to an aqueous solution. In some embodiments the sleeve cooperates with a container affixed to an advanced vehicle, such as unmanned aerial vehicle. In some embodiments, the sleeve is sterile.

In a first set of embodiments, a sleeve, shaped to an approximate shape of a target anatomical organ with an opening for inserting the target anatomical organ, includes a fabric that is porous with respect to an aqueous solution and has a tensile strength to hold the weight of the sleeve and of the target anatomical organ.

In some embodiments of the first set, the fabric of the sleeve may also include a different second opening for passing a blood vessel for the target anatomical organ. Further, the sleeve may be shaped to fit snuggly and envelop the anatomical organ. Further still, the fabric may be readily cut with surgical shears to access the anatomical organ. In other embodiments of the first set, the sleeve may further include, attached to the fabric of the sleeve, at least one of a temperature sensor and a vibration sensor.

In a second set of embodiments, a system for monitoring and transporting the anatomical organ includes a sleeve, shaped to an approximate shape of the target anatomical organ, comprising an opening for inserting the target anatomical organ and a fabric that is porous with respect to an aqueous solution and has a tensile strength to hold the weight of the sleeve and of the target anatomical organ; and a container, to hold the aqueous solution, the sleeve, and the anatomical organ. Further, the system includes a temperature sensor in thermal contact with the sleeve when the sleeve is inside the container. In some embodiments, the temperature sensor produces multiple temperature measurements at corresponding times. In yet another embodiment, the system comprises a wireless communication device in signal communication with the temperature sensor. Further still, the wireless communication device may transmit a first data based on the multiple temperature measurements at the corresponding times.

In some embodiments of the second set, the fabric of the sleeve may also include a different second opening for passing a blood vessel for the target anatomical organ. Further, the sleeve may be shaped to fit snuggly and envelop the anatomical organ. Further still, the fabric may be readily cut with surgical shears to access the anatomical organ.

Still, in other embodiments of the second set, the temperature sensor may be attached to the fabric of the sleeve. In yet another embodiment, the temperature sensor may be attached to the container.

In some other embodiments of the second set, the system includes a vibration sensor in mechanical contact with the sleeve when the sleeve is inside the container and in signal communication with the wireless communication device. In some embodiments, the vibration sensor produces multiple vibration measurements at corresponding times. Further, the wireless communication device may transmit second data based on the vibration measurements at the corresponding times. Further still, the vibration sensor may be attached to the fabric of the sleeve. In yet another embodiment, the vibration sensor may be attached to the container.

In yet some other embodiments of the second set, the system includes a global positioning system receiver to produce multiple position measurements at different times. Further, the global positioning system receiver is in signal communication with the wireless communication device. Further still, the wireless communication device may transmit second data based on the multiple position measurements at the corresponding times. In other embodiments, the global positioning system receiver may be attached to the container.

Still, in other embodiments of the second set, the system includes a barometric pressure sensor to produce multiple barometric pressure measurements at different times. Further, the barometric pressure sensor is in signal communication with the wireless communication device. Further still, the wireless communication device may transmit second data based on the multiple barometric pressure measurements at the corresponding times. In other embodiments, the vibration sensor may be attached to the container.

Further still, in other embodiments of the second set, the system comprises a processor and at least one memory including one or more sequences of instructions, wherein execution by the processor of the one or more sequences of instructions included in the at least one memory causes the system to receive the multiple of measurements from the temperature sensor and determine the first data, store the first data in the at least one memory, and transmit the first data using the wireless communication device.

In a third set of embodiments, an apparatus comprises a radio transceiver, at least one processor, and at least one memory including one or more sequences of instructions; wherein execution by the at least one processor of the one or more sequences of instructions included in the at least one memory causes the apparatus to receive metadata that indicates an anatomical organ, receive from the radio transceiver first data based on multiple temperature measurements taken at different times from a temperature sensor in thermal contact with the anatomical organ inside a container containing an aqueous solution, store in the at least one memory the first data in association with the metadata for the anatomical organ, determine output temperature data based on the first data and output metadata based on the metadata, and present the output metadata and the output temperature data on a display device.

In some embodiments of the third set, execution by the at least one processor of the one or more sequences of instruction included in the at least one memory causes the apparatus to receive from the radio transceiver second data based on multiple position measurements taken at different times from a global positioning receiver system in contact with the container configured to hold an aqueous solution and the anatomical organ, store in the at least one memory the second data in association with the metadata for the anatomical organ, determine output position data based on the second data, and present the output position data on the display device.

In other embodiments of the third set, execution by the at least one processor of the one or more sequences of instruction included in the at least one memory causes the apparatus to receive patient data indicating an electronic medical record for a transplant recipient of the anatomical organ, store in the at least one memory the patient data in association with the metadata for the anatomical organ, determine output patient data based on the electronical medical record for the transplant recipient, and present the output patient data on the display device.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a rendering that illustrates an example of a sleeve for monitoring and transporting an anatomical organ, according to one embodiment;

FIG. 2 is a photograph that illustrates an example of a container into which the sleeve of FIG. 1 is transported, according to one embodiment;

FIGS. 3A through FIG. 3C are renderings that illustrate various views of an example of the sleeve depicted in FIG. 5, according to one embodiment;

FIG. 4 is a block diagram that illustrates a sleeve for an anatomical organ, according to one embodiment;

FIG. 5A is a block diagram that illustrates a system for monitoring and transporting an anatomical organ wherein, at least one of the temperature sensor, the vibration sensor, the barometric pressure sensor, the global positioning system receiver, and the wireless communication device is inside the container, but no sensor is attached to the sleeve, according to one embodiment;

FIG. 5B is a block diagram that illustrates a system for monitoring and transporting an anatomical organ wherein, at least one of the temperature sensor, the vibration sensor, the barometric pressure sensor, the global positioning system receiver, and the wireless communication device is inside the container and at least one of the sensors is attached to the sleeve, according to one embodiment;

FIG. 6 is a block diagram that illustrates a system for monitoring and transporting an anatomical organ wherein, none of the sensors is inside the container, according to one embodiment;

FIGS. 7A through FIG. 7C are photographs that illustrates an example of the container depicted in FIG. 6, according to one embodiment;

FIG. 8 is a block diagram that illustrates a system for monitoring and transporting an anatomical organ in a UAV, according to one embodiment;

FIG. 9 is a block diagram that illustrates a system for monitoring and transporting an anatomical organ in a UAV, according to one embodiment;

FIG. 10 is a flow diagram that illustrates a method of transmitting a temperature measurement using the system described in FIGS. 5-9, according to one embodiment;

FIG. 11 is a flow diagram that illustrates a method of receiving and displaying data corresponding to an anatomical organ, according to one embodiment;

FIGS. 12A-12B are graphs that illustrate anatomical organ conditions stored and transmitted from the temperature sensor and vibration sensor during UAV flights, according to an embodiment;

FIGS. 13A-13B are graphs that illustrate anatomical organ conditions stored and transmitted from the altitude sensor and vibration sensor during UAV flights experiencing rapid ascent and descent, according to an embodiment;

FIGS. 14A-14C are graphs that illustrate anatomical organ conditions stored and transmitted from the vibration sensor, global position system receiver, and altitude sensor throughout several UAV flights, according to an embodiment;

FIG. 15 is a graph that illustrates anatomical organ conditions stored and transmitted from the vibration sensor during a piloted fixed wing, jet-powered, flight, according to an embodiment;

FIG. 16 illustrates a chip set upon which a portion of an embodiment of the invention may be implemented; and

FIG. 17 is a diagram of example components of a mobile terminal (e.g. handset) for communications, according to one embodiment.

### DETAILED DESCRIPTION

An apparatus and method are described for the monitoring and transport of an anatomical organ within a sleeve. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term“about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of kidney transplants transported on an unmanned aerial systems (UAS). However, the invention is not limited to this context. In other embodiments other organs may be the target anatomical organ. For instance, as non-limiting examples, some embodiments may include a heart, a lung, a spleen, and a pancreas, among others. It may also be noted, that the invention is not limited to human organs. In other embodiments, organs from animals may be the target anatomical organ. In other embodiments the organ is transported on other vehicles, such as manned aircraft, manned or unmanned surface land or sea vehicles or subsurface vehicles.

### 1. Overview

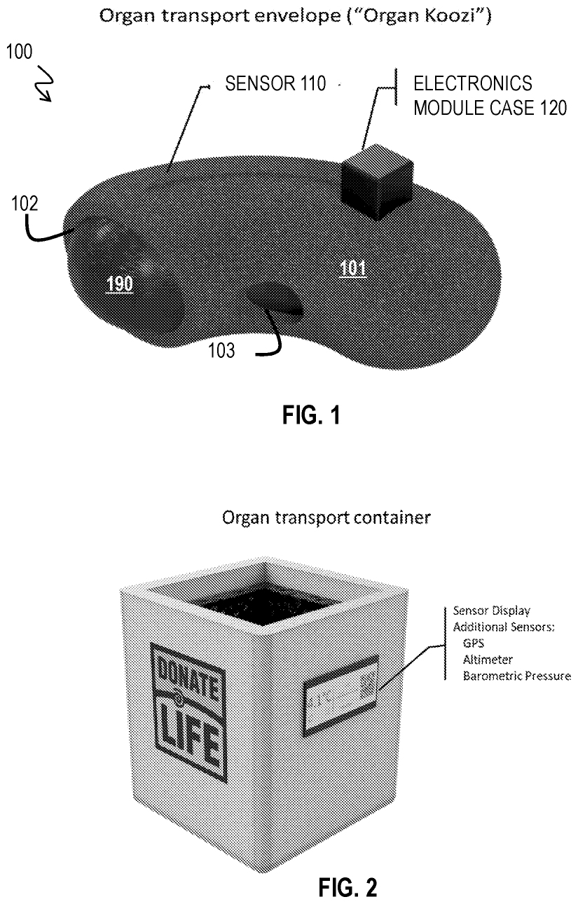

FIG. 1 is a rendering that illustrates an example of a sleeve assembly 100 for monitoring and transporting an anatomical organ 190, according to one embodiment. According to the illustrated embodiment, the sleeve assembly 100 includes a sleeve 101 that encloses a lumen shaped to approximate a shape of the target anatomical organ 190, either snugly, as shown, or loosely. In this context, snug refers to a fit around the target organ with no gaps and sufficient hold such that the organ does not slip uncontrollably relative to the sleeve when the sleeve is hand held. In some embodiments, the sleeve 101 does not approximate at all the shape of the anatomical organ 190, instead, the sleeve 101 largely surrounds and encloses the anatomical organ 190. The sleeve 101 has a first opening 102 configured for inserting the target anatomical organ 190 into the lumen of the sleeve. In some embodiments, the first opening 102 is resealable. The sleeve 101 is made of a fabric that is porous with respect to an aqueous solution and has sufficient tensile strength to hold a weight of the sleeve assembly 100 and a weight of the target anatomical organ 190. It is also advantageous if the fabric is sterilizable and does not damage the organ enclosed on contact with that organ. Any fabric that satisfies those requirements may be used and is easily discovered through routine experimentation. Example fabrics include cotton. In some embodiments neoprene (known to be sterilizable and safe on contact with kidneys and other organs) and other synthetics fabrics are used, and may be synthesized so as to have pores that allow the preservation fluid to permeate the fabric In embodiments in which the sleeve is loosely fitting, the sleeve material can allow circulation of preservation fluid inside the sleeve and the fabric of the sleeve need not be porous.

In another embodiment, the sleeve 101 is made of an impermeable fabric containing the aqueous solution. As it may be appreciated by those skilled in the art, the term “fabric” as used herein may also include materials produced processes other than weaving a thread, e.g. vulcanization or extrusion. In a non-limiting example, the sleeve 101 is a resealable polypropylene bag.

For purposes of illustration a target anatomical organ 190 is depicted as fitted snugly into the lumen of the sleeve 101, but the anatomical organ 190 is not part of the sleeve 101 or assembly 100. For purposes of illustration the target anatomical organ 190 in the illustrated embodiment is depicted as a single human kidney; but, in other embodiments, the sleeve 101 is shaped to enclose a different organ in the lumen, such as a lung, heart, pancreases, liver, eye, among others, as the target anatomical organ for a human or non-human organism.

The fit to the anatomical organ, whether snug or loose, is such that the sleeve can be grasped and held by a worker, such as a transporter, nurse or surgeon, without damage to, or loss of, the anatomical organ inside the sleeve during transport or during transplant surgery. The fabric is porous to an aqueous solution so that a preservation fluid used to sustain the organ in a state viable for transplant can contact the organ when the organ is inside the sleeve and the sleeve is immersed in the preservation fluid. Any preservation fluid known in the art may be used, such as University of Wisconsin solution (UW solution), Histidine-Tryptophan-Ketoglutarate solution (HTK solution), Euro-Collins solution, and Static Preservation Solution (SPS-1).

In some embodiments, the sleeve includes a different second opening 103. The second opening is placed relative to the first opening and sized so that one or more anatomical portions surgically attached to a patient during transplantation, such as veins, arteries and nerves (e.g., the portal vein for the kidney), can be fed through the second opening and surgically attached to a recipient subject while the anatomical organ 190 remains inside the lumen of the sleeve 101. This aids the surgical staff in holding the anatomical organ during an attachment procedure. After the anatomical portions are attached, the sleeve can be removed after cutting the sleeve on a path between the first opening 102 and the second opening 103. In such embodiments, it is advantageous for the fabric of the sleeve to have the property that it is readily cut with surgical shears that are commonly available in the transplant operating arena, at least on a path between the first opening 102 and the second opening 103. Thus, the sleeve can be used not only during transport but also during the attachment procedure.

In some embodiments, the sleeve also includes one or more sensor 110 that can be used to track the condition of the organ during transport. Example sensors include one or more of a temperature sensor, immersion sensor to ensure the sleeve with any organ inside has been immersed in the preservation fluid, a vibration sensor to track whatever possibly disruptive vibrations or pressures to which the sleeve and any organ inside has been subjected, an accelerometer to track whatever possibly disruptive movement or direction to which the sleeve and any organ inside has been subjected. In various embodiments, the one or more sensor 110 contact the outside of the sleeve, are embedded in the fabric of the sleeve, or penetrate to the surface of the lumen inside the sleeve to contact any anatomical organ inside the lumen of the sleeve. In some of any of these embodiments, the sensor terminates on an outer surface or outside the sleeve in a connection terminal for later connection to a power supply or information communication link or some combination.

In some embodiments, the sleeve assembly 100 includes an-immersion proof electronics module case 120 attached to an outside surface of the sleeve 101. The case encloses electronics for powering or operating or receiving or transmitting data from the one or more sensors 110, or some combination. In some embodiments the, electronics includes: a power supply; one or more sensors that do not require contact with the organ or sleeve, such as sensors for barometric pressure, humidity, ambient air or preservation fluid temperature, inertial measurements, geographic location (e.g., global positioning system, GPS, receiver); or, electronic components for data multiplexing, data conditioning/pre-processing, storage, retrieval, or communication with a local or remote processor; or, some combination. In some embodiments, the electronics module case 120 includes a port or cable (not shown) for attachment to some external system, such as a power supply, processor, or communications module, or some combination. In other embodiments, the sensors may be placed outside the sterile sleeve or bag containing the anatomical organ. While, in some other embodiments, the sensors are also placed in a sterile enclosure, either together or separate from the anatomical organ.

FIG. 2 is a photograph that illustrates an example of a container into which the sleeve system 100 of FIG. 1 is placed for transportation, according to one embodiment. The sleeve system 100 includes at least sleeve 101. The container is configured to hold preservation fluid and the sleeve 101 during transport in a vehicle, such as on an unmanned aerial systems (UAS). The container is also configured for thermal insulation to help stabilize the temperature of the preservation fluid and organ with or without heating or refrigeration to fall within a range of acceptable transportation temperatures. An example range of acceptable transportation temperatures includes from −2 degrees C. to 10 degrees C. In a non-limiting embodiment, the container is further configured to withstand forces and pressures in ranges between 0 to 5 g and 0 to 10 kPa, respectively. The container includes an opening for inserting the sleeve 101 or system 100 and any organ inside the sleeve into the container. In some embodiments, the container includes a lid configured to stay in place and prevent escape of the sleeve system 100 or sleeve 101 or preservation fluid during transport, such as during rough flying when the container might be subjected to inversion. For example, one embodiment of the container is a standard cardboard box enclosing an insulating box of polystyrene foam.

In some embodiments, the container includes additional electronic or optical modules, such as: a display device to present current or cumulative or extreme values produced by any of the sensors, or some combination; additional sensors, such as an altitude radar or laser sensor giving distance to the ground or other obstruction: or one or more of the sensors that would otherwise be in the electronics module case 120, as described above: or some combination. In some embodiments, the container includes a cable or port, on an inside or outside surface of the container, configured as a complementary terminal to connect to a connection terminal of any sensor 110 or electronics module 120 attached to the sleeve 100 or to any system external to the container but on the same transportation vehicle.

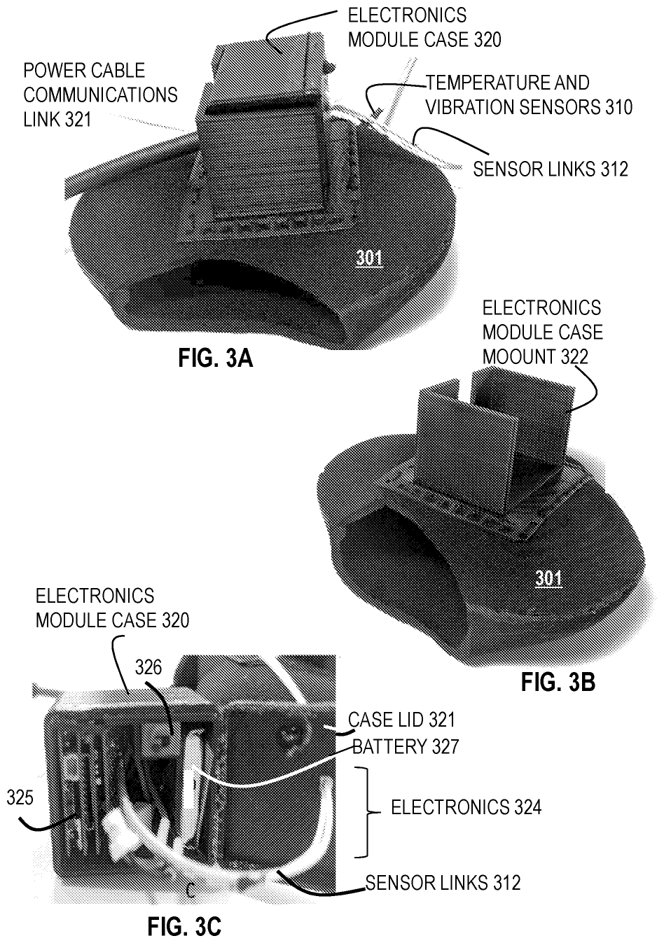

FIGS. 3A through FIG. 3B are renderings that illustrate various views of an example of the sleeve assembly depicted in FIG. 1, according to one embodiment. The depicted sleeve assembly includes a sleeve 301 and an electronics module case 320 with a power cord and communications link 321 and two sensor links 312 that extend outside the case 320 and connect to temperature and vibration sensors 310. As shown in FIG. 3B, an electronics module case mount 322 is attached to the fabric of the sleeve 301 and configured to attach and seal to the electronics module case 320.

The organ sleeve or “Koozi” 301 is a flexible, permeable, and disposable sleeve that, through light compression, ensures secure sensor contact with the sleeved organ. According to the illustrated embodiment, the organ sleeve is formed of a polyester covered neoprene foam. The sleeve is configured to fit the size and shape of an average human kidney; and having a single hole to allow insertion of the organ and free motion of essential fluid ducts (e.g., vasculature and ureter). The elasticity of the neoprene and polyester fabric coating permitted the ideal compression required for mechanical coupling of sensor modules. The open-cell structure of the neoprene and the porous polyester allows transmission of fluid and nutrients through the sleeve walls from the outer solution. The illustrated sleeve 301 is made of a fabric coated neoprene structure, custom cut and sealed with a neoprene contact cement. The illustrated sleeve 301 has a custom designed shape and size to accommodate the average human kidney.

As depicted in FIG. 3C, the illustrated sleeve assembly further includes a custom electronics system designed and assembled to allow continuous monitoring, recording, and transmission of temperature and vibration data acquired at the organ surface. The electronics package was developed to minimize size, weight, and power requirements while maintaining modularity for adaptability and future upgrades. The system and device are configured to stabilize electronics and sensor units and enable external power and communications interfaces using a custom water-resistant housing. The electronics and sensor units include a 4-stage vertical stack of 3 commercial components and 1 custom component. The electronics unit includes a processing stack having a microprocessor unit, an inertial measurement unit, and a data-logging unit. The electronics stack further includes communication connections and thermistor measurement circuitry for a 100 kiloOhm (kOhm) NTC thermistor. Data transfer is accomplished through serial communication via a 4-wire waterproof cable leading to the data transmission module. The illustrated embodiment is powered through a lithium ion battery included in the custom housing serving as the electronics module case 320. In the illustrated embodiment the case 320 is a custom 3D printed enclosure for water proof contact with a human organ during transport. The electronics 324 include sensor stack containing a microcontroller, SD card unit, accelerometer (vibration) unit, and thermistor circuitry on circuit board s 325. Links 312 connect the electronics 324 to the thermistor and vibration sensors 310. Power to the device is controlled through an externally accessible IP68 rated (dustproof, waterproof) power switch 326. A LiPo battery 327 provides power for independent function of electronics 324. The case includes a custom enclosure lid 321 that provides a seal to keep preservation fluid out of the electronics 324.

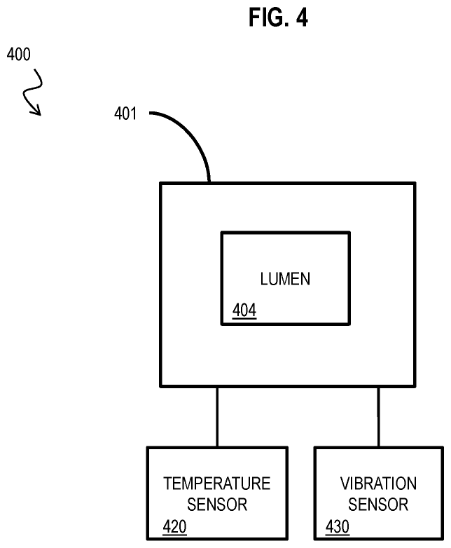

FIG. 4 is a block diagram that illustrates an example sleeve assembly 400 for transport of an anatomical organ, according to one embodiment. The assembly 400 includes a sleeve 401 with a lumen 404 that is configured to fit an anatomical organ. In the illustrated embodiment, the assembly 400 includes a temperature sensor 420 and vibration sensor 430 in thermal and mechanical contact, respectively, with the sleeve 401 or lumen 404.

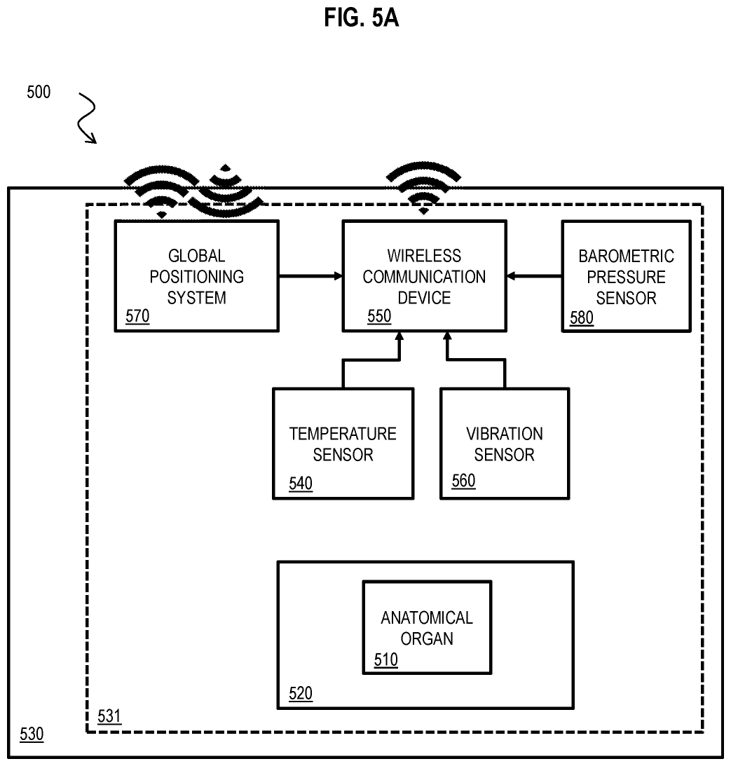

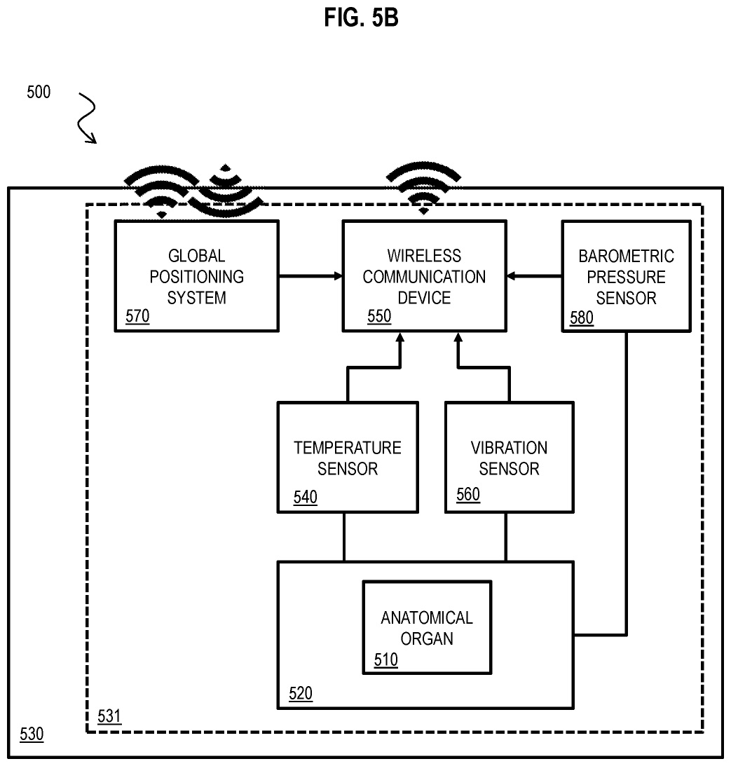

FIG. 5A is a block diagram that illustrates a system 500 for monitoring and transporting a target anatomical organ 510, according to one embodiment. The system 500 includes a sleeve 520, a container 530, a temperature sensor 540, and a wireless communication device 550 placed inside the container 530. In an embodiment, the temperature sensor 540 may be detached from the fabric of the sleeve 520, as illustrated in FIG. 5A, or attached to the fabric sleeve 520, as illustrated in FIG. 5B.

In an embodiment, the sleeve 520 includes a first opening for inserting the target anatomical organ 510. Although included for illustration of operation, the organ 510 is not part of the sleeve 520 or system 500. In another embodiment, the sleeve 520 may snugly envelop the target anatomical organ 510 without applying so much pressure that the anatomical organ 510 is compressed but it may not be loose enough that the anatomical organ 510 can freely shift and move about the sleeve 520. In a non-limiting embodiment, the sleeve 520 may include a fabric that is porous with respect to an aqueous solution 531. In a non-limiting example, the fabric may be neoprene. However, the fabric need only be porous with respect to the aqueous solution 531 and the specific material is non-limiting. In some embodiments, the material has enough tensile strength to hold a weigh of the sleeve and a weight of the target anatomical organ 510. In yet another embodiment, the fabric may be readily cut with at least one of scissors, shears, and surgical knives. Still, in other embodiments, the fabric may be readily dismantled by hand. In some other embodiments, the sleeve 520 may have a second opening. Further, the second opening may be used to pass a vessel (such as a blood vessel or ureter) for the target anatomical organ 510.

It may be appreciated by those skilled in the art that the term “anatomical organ” is intended to be non-limiting and may be any organ capable of being transported or transplanted. As non-limiting examples, the anatomical organ 510 may be a human kidney, a human heart, a human lung, a human spleen, and a human pancreas. It may also be appreciated, that the anatomical organ 510 may also be any organ, capable of being transported or transplanted, belonging to an animal.

Returning to FIG. 5A, in some embodiments, the sleeve 520 holding the anatomical organ 510, the temperature sensor 540, and the wireless communications device 550 may be placed in the aqueous solution 531 within the container 530. Further still, the temperature sensor 520 may be in thermal contact with the sleeve 520 when the sleeve 520 is inside the container 530. In other embodiments, such as the non-limiting embodiment shown in FIG. 5B, the temperature sensor 540 may be attached to the fabric of the sleeve 520 when the sleeve 520 is placed in the aqueous solution 531 contained within the container 530.

In yet another embodiment, the temperature sensor 540 may, either periodically or following an event, take a multitude of temperature measurements. Further, the temperature sensor 540 may be in communication with the wireless communication device 550 to wirelessly transmits a first data based on the multiple temperature measurements taken by the temperature sensor 540.

Returning to FIG. 5A, in some other embodiments, the system 500 may include at least one of a vibration sensor 560, a global positioning system receiver 570, and a barometric pressure sensor 580 in communication with the wireless communication device 550. In an embodiment, the at least one of the vibration sensor 560, the global positioning system receiver 570, and the barometric pressure sensor 580, as well as, the sleeve 520 containing the anatomical organ 510, the temperature sensor 540, and the wireless communications device 550 may be placed in the aqueous solution 531 within the container 530.

In an embodiment, the vibration sensor 560 may, either periodically or following an event, take a multitude of vibration measurements and, in communication with the wireless communication device, wirelessly transmits the second data based on the multiple vibration measurements. In other embodiments, the vibration sensor 560 may be either detached from the sleeve 520, as illustrated in FIG. 5A, or in mechanical contact with the sleeve 520, as illustrated in FIG. 5B. In some other embodiments, the vibration sensor 560 may be attached to the fabric of the sleeve 520.

Returning to FIG. 5A, in some embodiments, the global positioning system receiver 570 may, either periodically or following an event, produce multiple position measurements and, in communication with the wireless communication device Bx70, wirelessly transmits a second data based on the multiple position measurements.

In an embodiment, the barometric pressure sensor 580 may, either periodically or following an event, produce multiple barometric pressure measurements and, in communication with the wireless communication device Bx70, wirelessly transmits the second data based on the multiple barometric pressure measurements.

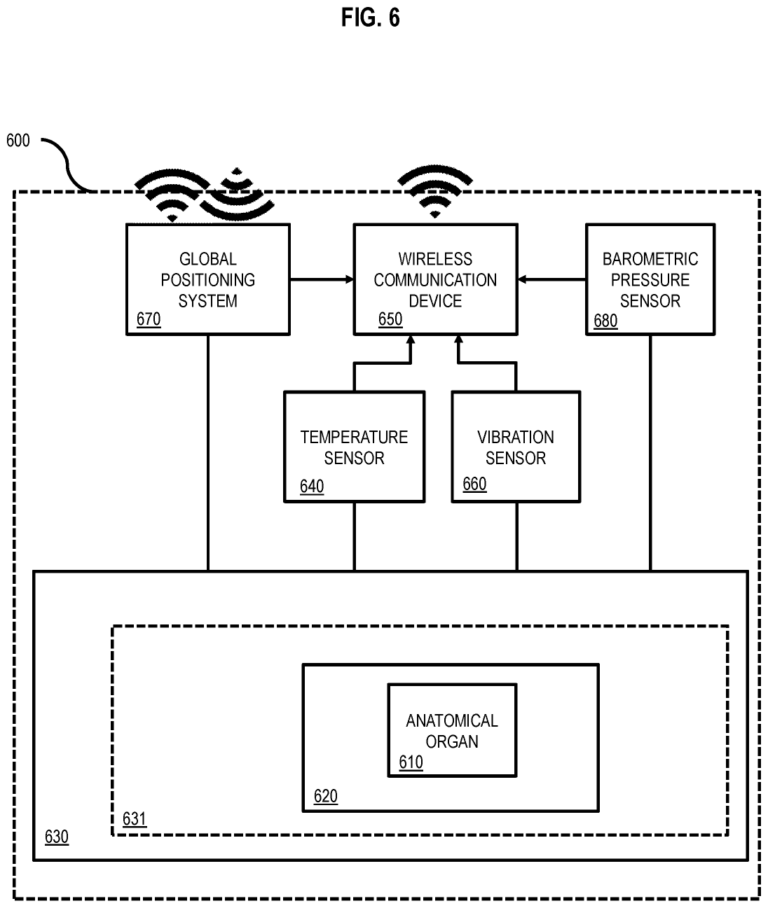

FIG. 6 is block diagrams that illustrates a system 600 for monitoring and transporting a target anatomical organ 610, according to one embodiment. FIG. 6 is identical to FIG. 5A, except that the temperature sensor 640, the wireless communication device 650, the vibration sensor 660, the global positioning system 670, and the barometric pressure sensor 680 are outside of the container 630. In another embodiment, at least one of the temperature sensor 640, the wireless communication device 650, the vibration sensor 660, the global positioning system 670, and the barometric pressure sensor 680 are not attached to the container 630.

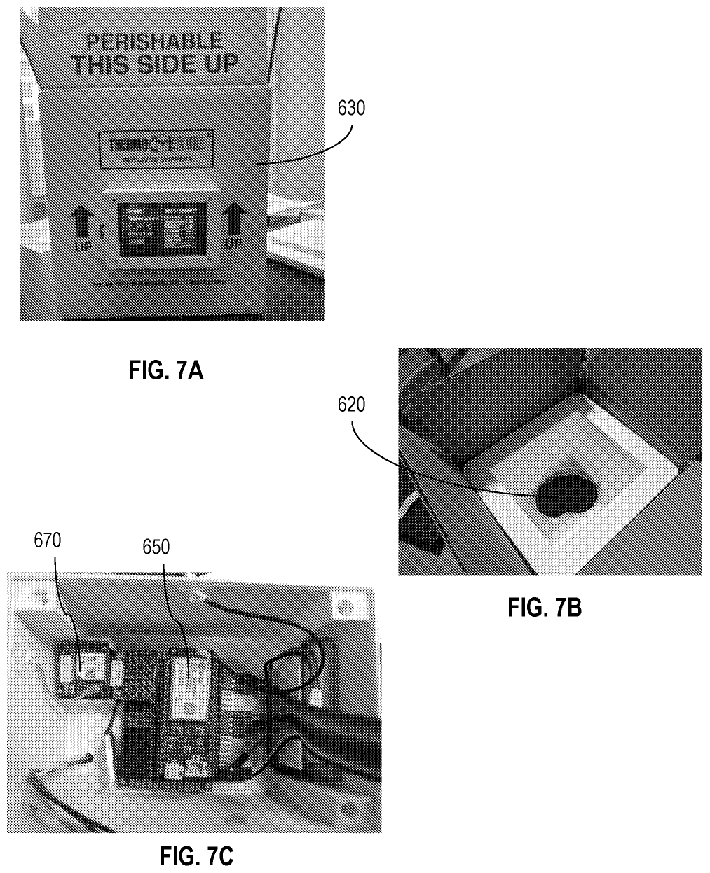

FIG. 7A through FIG. 7B are photographs that illustrates an example of the container depicted in FIG. 6, according to one embodiment. FIG. 7B shows the sleeve 620 inside the container 630 while the wireless communication device 650 and the global positioning system 670 are attached to the outside of the container 630—shown in FIG. 7C.

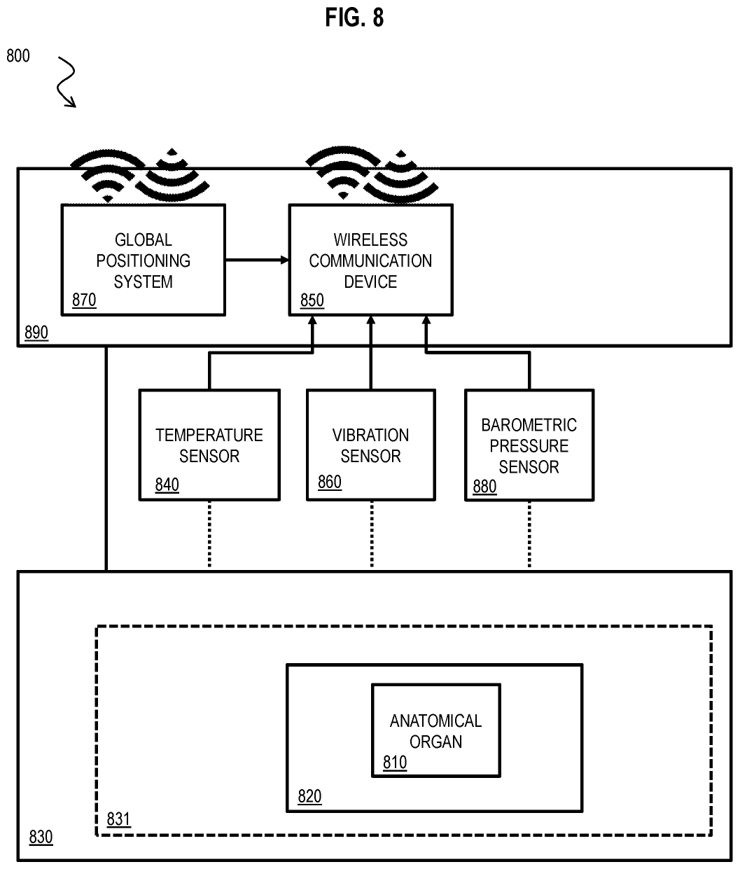

FIG. 8 is a block diagram that illustrates a system 800 for monitoring and transporting an anatomical organ 810 in a UAV 890, according to one embodiment. The system 800 includes a container 830, a temperature sensor 840, at least one of a vibration sensor 860 and a barometric pressure 880, and a UAV 890. In an embodiment, the sleeve 820 containing the anatomical organ 810 is placed inside the container 830. Further, the container 830 is mechanically attached to the UAV 890. In another embodiment, the UAV 890 includes a wireless communication device 850. Further, the UAV 890 may include a global positioning system receiver 870 in communication with the wireless receiver 850. Further still, the wireless communication device 850 may receive a third data relating to the controls and guidance of the UAV 890.

In yet another embodiment, temperature sensor 840 may, periodically or following an event, take multiple temperature measurements and, in communication with the wireless communications receiver 850 transmit the first data based on the multiple temperature measurement. In other embodiments, at least one of the vibration sensor 860 and a barometric pressure 880 may, periodically or following an event, take multiple vibration measurements and multiple barometric pressure measurements, respectively, and, in communications with the wireless communication device 850, transmit the second data based on at least one of the multiple vibration measurements and multiple barometric pressure measurements. In still other embodiments, the temperature sensor 840, and at least one of the vibration sensor 860 and the barometric pressure sensor 880 may or may not be attached to the container 830.

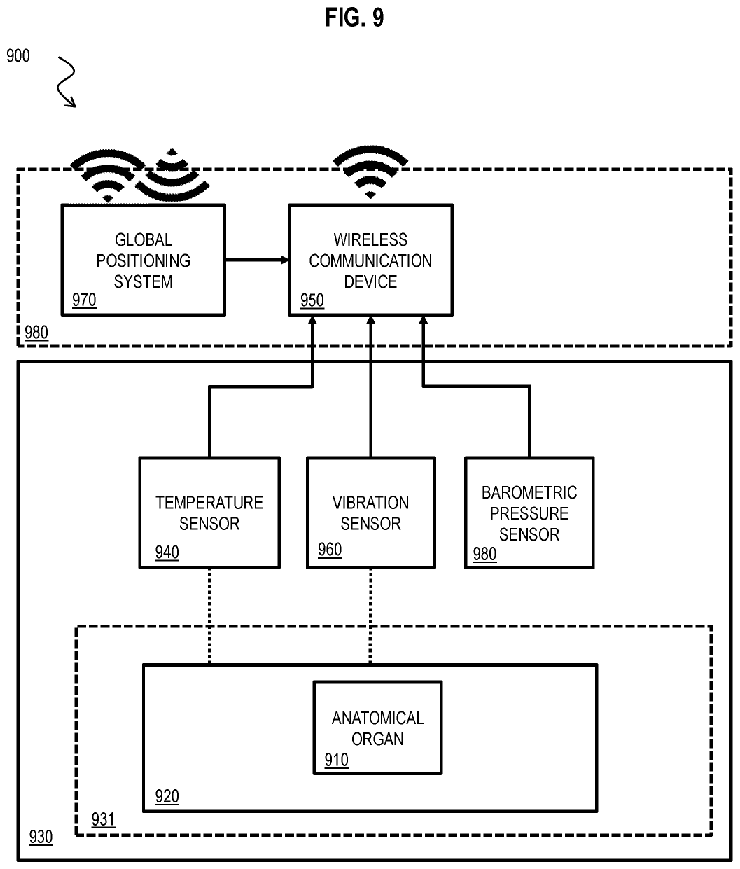

FIG. 9 is a block diagram that illustrates a system 900, according to one embodiment, for monitoring and transporting an anatomical organ 910 in a UAV similar to system 800 except that the temperature sensor 940, and at least one of the vibration sensor 960 and the barometric pressure sensor 980 are contained within the container 930 and may or may not be attached to the sleeve 920.

Although processes, equipment, and data structures are depicted in FIG. 4 through FIG. 6 and FIG. 7 though FIG. 9 as integral blocks in a particular arrangement for purposes of illustration, in other embodiments one or more processes or data structures, or portions thereof, are arranged in a different manner, on the same or different hosts, in one or more databases, or are omitted, or one or more different processes or data structures are included on the same or different hosts.

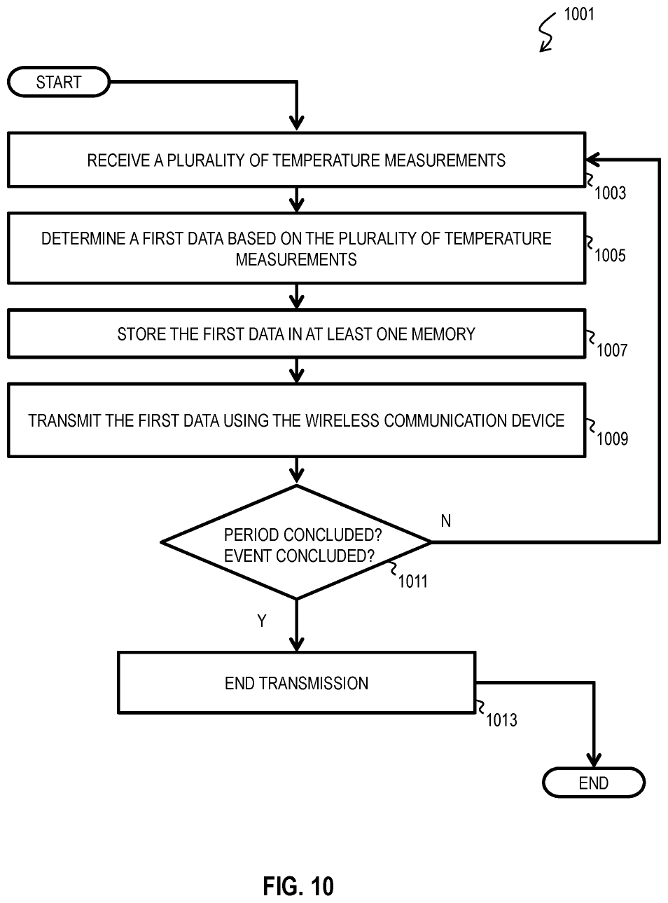

FIG. 10 is a flow diagram that illustrates a method of transmitting a temperature measurement using the system described in FIGS. 5-9, according to one embodiment. The system 500 includes at least one processor and at least one memory including one or more sequences of instructions. In step 1003 the processor receives multiple temperature measurement from the temperature sensor. Then, in step 1005 the processor determines a first data based on the multiple temperature measurements from the temperature sensor. In step 1007, the processor stores the first data in the at least one memory, and in step 1009 it transmits the first data using the wireless communication device 550. If either transmission period indicating that no more first data is available, or an event concludes then wireless communication device 550 ends transmission in step 1013. If more data is available, then the process starts again.

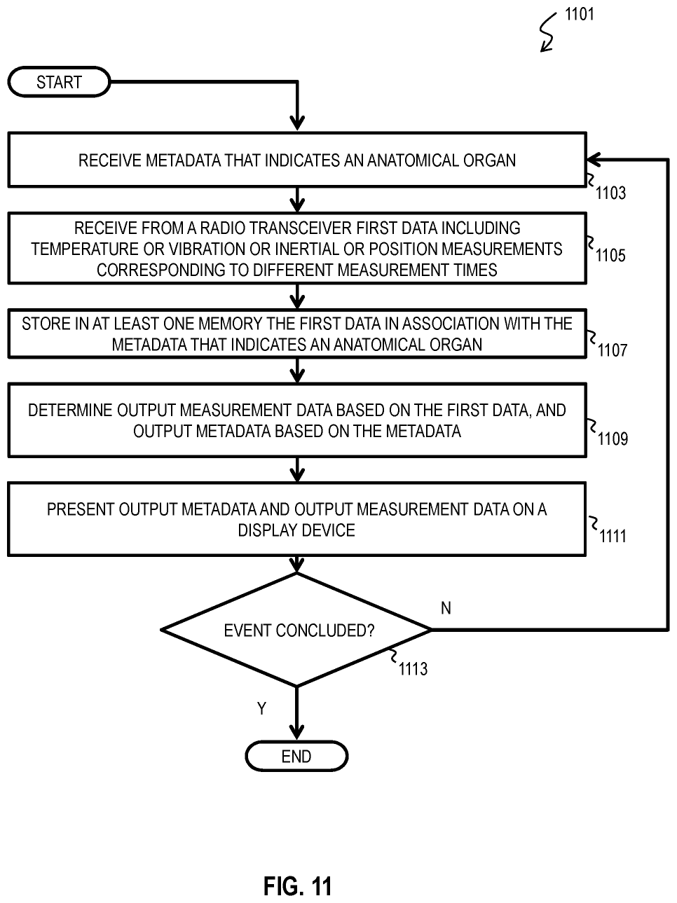

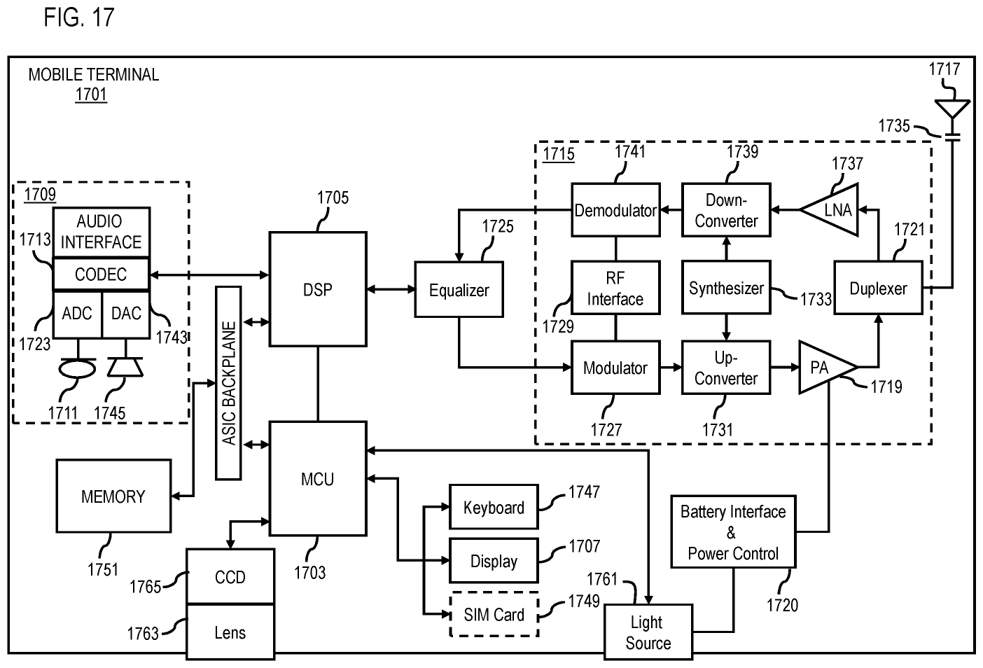

FIG. 11 is a flow diagram that illustrates a method 1101 of receiving and displaying data corresponding to an anatomical organ using the system described in FIG. 17, according to one embodiment. The mobile terminal 1701 includes a radio transceiver 1715, at least one processor 1705, at least one memory 1751, and a display device 1707. FIG. 17 is discussed in more detail below.

Returning to FIG. 11, in step 1103 the processor 1705 receives from the radio transceiver 1715 metadata indicating an anatomical organ. In an example embodiment, the anatomical embodiment may be the anatomical organ 190 illustrated in FIG. 1. Next, in step 1105, the processor 1705 receives first data from the radio transceiver 1715. First data may include multiple temperature, vibration, inertial, barometric pressure, or position measurements corresponding to different measurement times. In an embodiment, the first data includes temperature measurements corresponding to data reported by a temperature sensor in thermal contact with an anatomical organ inside a container configured to hold an aqueous solution. In step 1107, the processor 1705 stores, in the at least one memory 1751, the first data in association with the metadata that indicates an anatomical organ.

Then, in step 1109, the processor 1705 determines output temperature based on the first data, and output metadata based on the received metadata indicating an anatomical organ. The processor 1705, in step 11011, presents the output metadata and output temperature data to a user using the display device 1707. If an event occurs indicating the end of the first data or metadata, then the process ends, and no new output metadata or temperature data is updated. If more data is available, then the process starts again.

Although steps are depicted in FIG. 10 and FIG. 11, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

### 2. Example Embodiments

The HOMAL (Human Organ Monitoring and Quality Assurance Apparatus for Long-Distance Travel (HOMAL; patent pending) is an embodiment configured to measure temperature, barometric pressure, altitude, vibration, and location via global positioning system (GPS) during transportation, such as discussed above. These parameters are selected as they are perceived to be material during non-pressurized UAS transportation. However, other parameters—equally material and not mentioned herein—may capture the conditions and forces sustained by anatomical organ during non-pressurized transportation. A human kidney was used as an example anatomical organ in an experimental example embodiment of HOMAL.

The experimental HOMAL included a neoprene exoskeleton which gently encases the kidney. Embedded in the exoskeleton is a biosensor which measures each of the desired parameters in real time. These data are streamed every 10 seconds to a land-based server using wireless technology. The server data then auto-populate the “organ transplant monitoring system,” or OTMS, as an application (“app”) accessible on any standard internet-based device (e.g., mobile phone, computer, etc.). In some embodiments, a system includes the app running on the internet device.

The HOMAL communicates with a Smart Cooler as the container. The Smart Cooler has a graphical user interface (GUI) that allows the user to observe, for example, the real time temperature, acceleration, altitude, barometric pressure, intensity of vibration (versus frequency), latitude, longitude, Smart Cooler battery life, and wireless signal strength.

The test kidney donor was a 57 y/o African-American male with a history of HTN, alcoholism, and splenectomy for trauma in the remote past. The kidney donor profile index (KDPI) was 70 % and the donor was cytomegalovirus (CMV)+ as well as public health service (PHS) increased risk for social behavior. The donor was non-oliguric and was brain dead. The admission creatinine was 0.9 mg/dL, the peak creatinine was 0.9 mg/dL and the terminal creatinine was 0.5 mg/dL. At the time of recovery, there was scar tissue between the left kidney and the pancreatic tail, suggestive of prior pancreatitis.

The kidney arrived in a box which measured 27.9 cm×38.1 cm×22.86 cm. The weight of the shipped box inclusive of shipping containers/materials, University of Wisconsin (UW) solution, and organ was 5.1 kg. The kidney was uninjured and normal in appearance. The organ was stored in UW solution. The kidney failed to place nationally, and thus it was offered for research. The total cold ischemia time (CIT, which is the time period between organ explant and implant during which time the organ is cooled—in some instances the organ is cooled on ice to 4 degrees Celsius—and for which there is a limit if the tissue or organ is to be transplanted) at allocation was 19.0 hours. The total CIT prior to UAS testing (“box open”) was 63.3 hours. The kidney was shipped to our laboratory by a series of couriers and by commercial aircraft over a distance of 1060 miles. The kidney was cold-stored in UW solution for the entirety of shipment and testing. No damage to the kidney occurred in transit.

The kidney was 11cm×5 cm. There was a single artery, a single vein, and a single ureter. Aortic and arterial plaque were present. A post-recovery kidney biopsy obtained prior to shipping showed 12% glomerular sclerosis. The biopsy also showed focal, mild interstitial fibrosis and focal, mild arterial and arteriolar damage.

As additional CIT elapsed between allocation and testing, the kidney was re-biopsied immediately prior to UAV testing. After 4.5 hours of testing (including 1 hour and 2 minutes of UAV flight) the organ was biopsied a third time. The biopsies were stored in formalin and fixed in paraffin blocks. Hematoxylin and Eosin (H&E) stains were performed and the results were interpreted by a senior renal transplant pathologist at the University of Maryland.

Two thermometers were utilized to measure the organ. The HOMAL's thermistor was silicone tipped for efficacy in conductive solution. A second digital meat thermometer (Bradshaw International, Rancho Cucamonga, Calif.) was used to measure the core temperature of the kidney, the ambient air, and the UW solution. The second thermometer had a dual protective sheath, and a non-slip silicone head which was used to puncture the kidney. The second thermometer has a manufacturer tested range of −50 degrees Celsius to 300 degrees Celsius. The digital second thermometer was powered by a single L1154 Alkaline cell battery. Manufacturer instructions indicated that 20 seconds are required for temperature equilibration. In this study a wait greater than 30 seconds was performed to ensure accurate measurements. The meat thermometer allowed correlation of the temperatures measured by the HOMAL. All temperature measurements were taken five times and separated by 30 seconds. This was done to understand the variability of each device, and to enhance accuracy. HOMAL-derived barometric pressure data were quantified in milli-bar (1 millibar=10 kilopascals; kPa). Pressure units were then converted to kPa as this is the SI unit for pressure (atmospheric pressure is 100 kPa). HOMAL vibration was measured in acceleration units, e.g., meters per second per second (m/s²), not in hertz.

Latitude and longitude were recorded by the HOMAL, as described below. These data were provided by a standard global positioning systems (GPS), as is frequently found in cellular telephones. These were reported to the user by real time digital mapping once downloaded from a ground-based server.

Flights took place at the UAS test facility in southern, MD. For the present experiment, all flights and arrangements were managed by professional, fully-trained unmanned aerial systems (UAS) pilots in collaboration with the experiment lead. Two UAV's or “drones” were used; a primary drone carried the organ payload, and a secondary drone (a “chaser”) was utilized as a safety measure. The secondary drone also allowed for video data collection of the primary drone, as well as, quick identification of a crash site in case the payload drone crashed.

The primary UAV was a DJIM600 Pro. This device contains 6 vertically oriented motors which function by battery power. Each of the 6 motors was immediately beneath each of the rotors. This is advantageous because the payload was not in direct contact with potentially warm motors. The DJIM600 uses a warm up period of approximately 5 minutes prior to active flight. During this time the drone batteries are warmed from ambient temperature to a goal temperature of >25.0 degrees Celsius. This particular drone can manage a payload of approximately 9.1 kg (201 lbs). The drone is considered flight worth in wind speeds of up to 32.2 km/h (20 mph). A GoPro camera was mounted to the underside of the UAV for video data collection, and to visualize real time, the status of the organ.

The secondary drone was a DJI Inspire 1. This drone has 4 vertically oriented rotors, and an inferiorly mounted GoPro camera for video data collection. This drone was not designed to carry a payload beyond a simple camera.

Prior to active flight a formal pre-flight Operational Readiness Review (ORR) briefing was held to determine appropriateness of local weather patterns. The Federal Aviation Administration's (FAA) Automated Weather Observation Service (AWOS) at Saint Mary's Airport (WX AWOS-3) in Leonardtown, Md. was utilized. Personnel was identified, and specific roles were assigned. Safety measures were discussed. The organ donor and his family were acknowledged.

During the experiment there was data recordation by three sources: 1. the HOMAL device itself, 2. manual recordation during tests, and by 3. the DJIM600 drone. HOMAL data were saved real-time to onboard digital memory. For this experiment a secure digital (SD) card was used. This SD card functioned analogously to a “black box”—as is standard in commercial flight. HOMAL data were also loaded to the ground-based server every 10 seconds and recorded to a comma separated values (CSV) file. Each mission was punctuated by a time-stamp. CSV files were then accessed and analyzed in Microsoft Excel Professional Plus 2016. Additional statistics were analyzed using International Business Machines (IBM), SPSS version 25. Because of variations in mission parameters between missions, data were normalized such that temperature, vibration, and pressure could be compared between missions.

Prior to the experiment, the kidney was shipped in a sterile cylindrical plastic container filled with UW solution, per standard practice. Outside the cylindrical plastic container were two additional sterile organ bags, also per standard practice. Outside of these two sterile organ bags was a combination of nonsterile ice and water. The nonsterile ice and water were housed in a plastic-lined Styrofoam cooler, fit to the dimensions of the cardboard shipping box.

a) Pre-Flight Measurements.

Kidney temperature readings were taken indoors, where the ambient temperature was 19.6 degrees Celsius. The mean temperature of the nonsterile fluid external to the kidney was 3.3 degrees Celsius (SD 0.00). The UW solution was 0.9 degrees warmer (mean 4.2 degrees Celsius, SD 0.07) than the non-sterile ice water (p<0.001). The mean kidney core temperature was 5.8 degrees Celsius. The inferior pole of the kidney was warmer than the upper and middle poles (p<0.05). Temperatures of the upper and middle poles were not different (p>0.05). During temperature measurements, the interior pole of the kidney was oriented up, exposing it to ambient temperatures to a greater degree than the middle or upper poles.

b) Organ Preparation and Loading

Next, the organ was prepared and loaded into the HOMAL device. Organ preparation, consisting of removing perinephric fat from the kidney, lasted 5 minutes. Placement of the organ in the HOMAL and the artery lasted <10 seconds to successfully place. Vein and ureter were unaffected by the HOMAL. HOMAL temperatures were then correlated with the UW solution in which the HOMAL was submerged. The mean temperature recorded by the HOMAL was warmer than the UW solution by 1.1 degrees Celsius. The kidney-HOMAL unit was then packaged in the Smart Cooler (container) for transportation. A temperature decrease to a mean of 3.9 degrees Celsius was observed prior to active flight. Over approximately 1 hour, the HOMAL showed a stable temperature of 2.5 degrees Celsius.

c) Ambient Out-Of-Doors Measurements

Ambient out-of-doors measurements as reported by the FAA AWOS were as follows: flight time temperature was 5.0 degrees Celsius, visibility of 16.1 km (10.0 miles), and “clear” sky conditions. Winds speeds were 17-26 km/h (9-14 knots). These data were considered favorable.

d) Flight Experiment

A total of 14 UAS missions were performed. Vibration and barometric pressure changed with motion and altitude. Latitude and longitude varied as anticipated. Increases in vibration intensity were noted only mildly when the motors were activated. Missions reported in results sections 4a and 4b (below) took a total of 26 minutes of flight time.

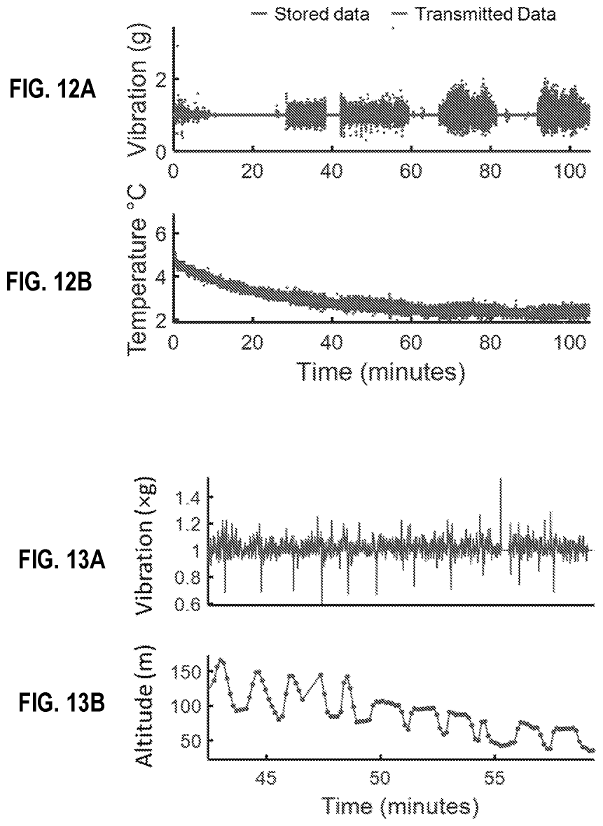

Up-and-Down. First, a series of take-off and landing missions (n=5) were performed. With the organ as payload, the UAV was directed to take off, and accelerate to 1.5 m/s to a maximum height of 61 meters (200 feet). At 61 meters, the drone was visible, but the organ transport box was not easily visualized from the ground with the naked eye. Temperature was stable (FIG. 12A). Up-and-down motion was associated with vibration changes. Vibration changes did not exceed 0.5 G (FIG. 12B). We observed a decrease in barometric pressure by 0.8 kPa when the kidney reached maximum altitude.

Hover. Next, a series of hover missions (n=5) were performed. The drone was directed to take off and accelerate at 1.5 m/s to an altitude of 30.5 meters (100 feet). At 30.5 meters, the organ payload was more clearly visible. During each of 5 hovers, there were no concerns for organ safety and wind speed did not seem to affect drone and/or organ stability. During hover, temperature remained stable (FIG. 12B). Vibration ranges were similar to those observed in up-and-down flights and were all less than 0.5 G (FIG. 12A). Pressure changes were approximately 1-half (0.4 kPa) those observed in up-and-down missions, reflecting the association between higher altitudes and lower barometric pressure.

Comparison to Traditional Flight. A series of distance missions (n=4) were performed. Each distance experiment included a flight of >762 meters (2500 feet) at an altitude of 122 meters (400 feet). These missions were modeled after the potential shipment of donor organs between inner city hospitals. Mission 1 and 2 took a combined 14 minutes and 22 seconds. The maximum speeds were 38 mph and 30 mph for missions 1 and 2, respectively.

The drone was grounded between flights 2 and 3, during which time the batteries were changed to allow for additional distance testing. Differences in speed were driven by wind speed. Missions 3 and 4 took a combined 12 minutes and 9 seconds. Maximum speeds were 41 mph and 42 mph for missions 3 and 4, respectively. The maximum travel distance was at the limit of line-of-site, (experiment 3), during which the kidney was transported outbound 2415 meters (7924 feet, 1.50 miles), for a total of 4830 meters (15,848 feet, 3.0 miles).

A standard fixed wing flight on a dual engine turboprop King Air was performed as a control for organ drone transportation. Organs are typically cold stored and transported by airplane. The difference between the fixed wing airplane and the UAV was that the fixed wing airplane has a pressurized cabin. Thus, the primary comparison between standard flight and UAV flight is vibration. The flight lasted 28 minutes. Fixed wing flight was associated with changes in vibration of >2.0 G. Significantly more vibration was observed during takeoff and landing with a fixed wing aircraft than with drone transportation difference in the vibration intensity or pressure with air travel when compared to drone travel (p<0.001). More specifically, the organ experienced more vibratory events in fixed wing aircraft at takeoff and landing, than it did with drone transportation at any time during its flight. However, once airborne, little vibration was experienced by the kidney.

FIGS. 12A-B are graphs that illustrate anatomical organ conditions stored and transmitted from the temperature sensor and vibration sensor during UAV flights, according to an embodiment. Blue markers represent data logged on the local memory (SD storage). Orange markers represent data transmitted through the wireless module. FIG. 12A represents vibration data recorded from outside the container. FIG. 12B represents temperature data measured from the organ surface. Vibration data in FIG. 12A visibly indicates flight periods and rest periods, where values continuously resting at 1.0×g indicate a resting state or smooth, constant-velocity flight, in which gravity (1×g) is the only acceleration measured. When compared with position recordings, periods of intense vibration correspond directly to active UAV flight periods.

Data stored directly on the organ sensor module, shows high-resolution recordings of both temperature and vibration. Transfer to the wireless communications device is slowed by sequential processes that greatly reduce the time resolution. FIGS. 12A and 12B compares the data logged directly at the organ module (˜7.5 Hz), “Stored Data,” and data transmitted to OTMS (−0.11 Hz), “Transmitted Data.” Results indicate that the slower data rate does lead to some data loss; however, the transmitted data provides sufficient indication of vibration and temperature conditions to inform an observer on the remote mobile device of unusual conditions during flight.

FIG. 13A through FIG. 13B are graphs that illustrate anatomical organ conditions stored and transmitted from the altitude sensor and vibration sensor during UAV flights experiencing rapid ascent and descent, according to an embodiment. Comparison of vibration logs with altitude logs, allowed correlation of plot features with flight events. For example, ascent/descent cycles showed periodic features in the vibration data (see FIGS. 13A and 13B). Sharp vertical changes in vibration intensity likely indicate rapid ascent or free-fall. When compared to altitude measurements acquired from the container, the vibration measurements clearly indicate rapid ascent and descent events. Each direction change corresponds to a prominent peak in the vibration graph. The direction of the peak (up or down) directly implies the direction of the altitude change. Simply, as the organ experienced a constant velocity ascent there would be little or no acceleration (i.e. only drone induced vibration), then as the ascent ended the slowing/stopping creates a measurable downward acceleration.

While the demonstration was ultimately successful, it was observed that the thermistor element utilized for this experiment was incompatible with full submergence in the ionic organ maintenance fluid used to preserve biological function during transport. In other embodiments, the thermistor was configured to operate stably in the electrolyte solution (e.g., electrical environment). For example, in some embodiments, the system includes shielding formed of liquid neoprene rubber and polyvinylchloride heat shrink tubing. Further still, some other embodiments include using a commercially available waterproof temperature sensor.

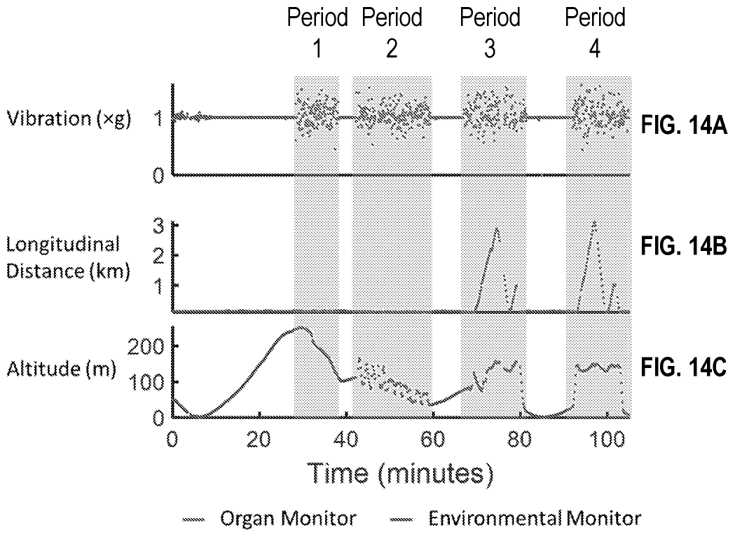

FIGS. 14A through 14C are graphs that illustrate anatomical organ conditions stored and transmitted from the vibration sensor (FIG. 14A), global position system receiver (FIG. 14B), and altitude sensor (FIG. 14C) throughout several UAV flights, according to an embodiment. Four distinct segments of flight were observed and denoted as Periods in FIGS. 14A through 14C. Periods 1-4 represent high vibration periods occurring in various flight pattern tests; Period 1: vertical takeoff and rapid descent, Period 2: vertical takeoff and rapid ascent/descent cycles, Period 3 & 4: vertical takeoff and long-distance (3 km) transport test.

Together, these data illustrate the feasibility of recording meaningful flight metrics and correlating measurement fluctuations with flight events. The relatively low sampling rates associated with the communications protocol in this particular experiment provided some control on the depth of analyses possible with the current data set. It may be appreciated that other embodiments may use higher data transfer rates or local storage.

In the present experiment, the global positioning system receiver included in the sensor module enables simple global positioning of the tracked package. During the experiment, a small low-cost global positioning system receiver allowed robust location of the package across a 3 km distance. It may be appreciated that data collected and reported by the global positioning system receiver and either transmitted using the wireless communications device or recorded internally in the memory can be graphed using commercial off the shelf software. This data combined with altitude data—potentially gathered from a barometric pressure sensor—may be used to illustrate the travel paths throughout the duration of the tests.

Additionally, control experiments were performed where the HOMAL was further loaded onto a small, piloted, fixed wing jet aircraft. During this experiment test interference from the piloted aircraft prevented transmission of data from the HOMAL. In addition, package preparation damaged the organ thermometer. As a result, only vibration data from the organ module could be collected and processed.

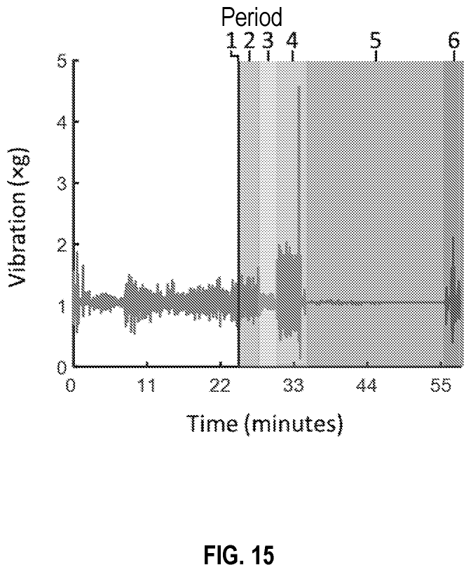

FIG. 15 is a graph that illustrates anatomical organ conditions stored and transmitted from the vibration sensor during a piloted fixed wing, jet-powered, flight, according to an embodiment. Vibration data from the flight was used to identify particular events throughout the flight denoted as Periods 1-6: start (1), boarding (2), taxiing (3), take-off (4), flight (5), and landing (6). Each of these periods induced vibration patterns that are not only distinct from other flight periods, but from data gathered during UAV flights. Whereas moderate vibration intensity was observed in the UAV throughout the longitudinal flights, large vibrations were observed in the fixed wing aircraft only during takeoff and landings and was otherwise observed to be relatively calm during longitudinal flight.

e) Kidney Status After Drone Transport

The kidney was anatomically normal after UAV flight testing. Total on-board drone time was 1 hour and 2 minutes. The HOMAL was intact and there were no signs of HOMAL-associated organ injury. Biopsies were taken prior to and immediately after drone flights. Drone flight did not affect biopsy results. Prior to and after drone flight, there was 11-12% glomerular sclerosis, cortical scarring, and hyalinosis.

Despite the dramatic improvement in organ transplantation outcomes over the last several decades, there remains a woeful disparity between the number of recipients on the organ transplant waiting list and the total number of transplantable organs. Indeed, this problem has fueled interest in organ regeneration, 3D organ printing, and xenotransplantation, however, these technologies are many years away from clinical implementation.

Drone organ transportation could widen the donor organ pool and allow for more organ transplants. Because organ quality is higher when CIT is low and because higher quality organs result in more life-years for the recipient, lower CITs resulting from UAS transport could add life-years to transplant recipients. Patients who receive a higher quality transplant are less likely to require a re-transplant, allowing another patient on the waiting list to undergo transplantation. Also, if organs could travel more efficiently, surgeons would be more likely to accept organs, particularly those considered marginal. Lastly, if the OPOs around the United States had the ability to move organs more quickly, they may entertain the use of many donors who are not currently considered candidates.

The mean rate of discarded kidneys in the United States is approximately 20%. Indeed, many of these kidneys may have been usable were CITs expedited. Based on a value of 20% and a transplant volume of 13,501 deceased donor kidneys in 2016, as many as 2700 kidneys may have been available for transplantation were CIT minimized By way of example, a recent study showed that for 5,000 randomly selected—declined kidney offers, patients were more likely to be alive if the offers been accepted versus declined. These data suggest that if tools such as the organ monitoring device discussed above and perhaps drone transportation were capable of shifting the balance of information in favor of transplantation, more patients could be transplanted with currently available resources.

Potential drone take-offs and landings directly at transplant hospitals, may translate into organ transportation where reduced CIT would be only a factor of the speed of the drone and the time it takes to package the organ. For instance, if an organ drone could travel 350 miles per hour, an organ in Los Angeles could arrive in Baltimore (2645 miles) in 7.5 hours. Similarly, an organ in New York, it could arrive in Baltimore (192 miles) in 33 minutes. For comparison, the national average CIT is 16-18 hours, including local and national sharing. Difficult-to-reach areas of the country, such as southern Florida, have markedly longer average CITs. In some regions CITs routinely exceed 30 hours for kidneys.

Advantages of the methods and systems discussed herein in the context of organ transplantation, could not only increase but also streamline the flow of data to the stakeholders. For instance, the status and location of accepted organs would be accessible by merely opening an application (such as the OTMS) on the user's cell phone. As designed, the example embodiments discussed above reliably provided real time organ status and location. These data were downloaded to a ground-based server and stored. These data were also available on the mobile phone of each of the project team members in real time. This is exciting because, at present, time sensitive organ transplants are not monitored by GPS. Currently, obtaining organ status and location updates involves multiple phone calls with busy couriers.

### 3. Hardware Overview

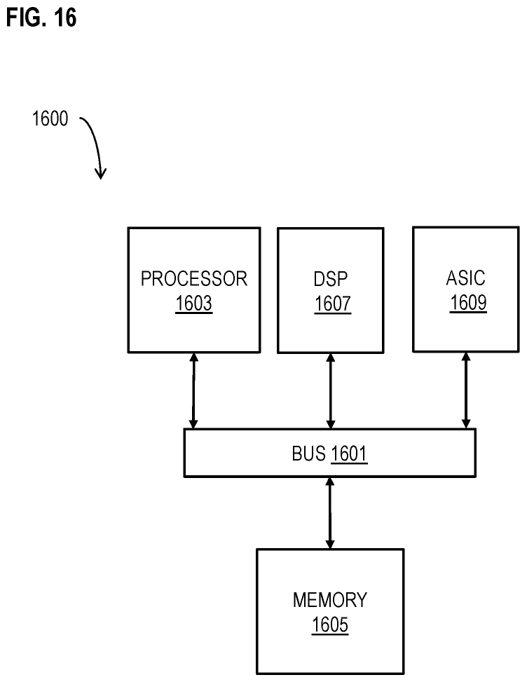

FIG. 16 illustrates a chip set 1600 upon which an embodiment of the invention may be implemented. Chip set 1600 is programmed to perform one or more steps of a method described herein and includes, for instance, the processor and memory components described with respect to FIG. 10 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1600, or a portion thereof, constitutes a means for performing one or more steps of a method described herein.

In one embodiment, the chip set 1600 includes a communication mechanism such as a bus 1601 for passing information among the components of the chip set 1600. A processor 1603 has connectivity to the bus 1601 to execute instructions and process information stored in, for example, a memory 1605. The processor 1603 may include one or more processing cores with each core configured to perform independently. A multi-core processor enables multiprocessing within a single physical package. Examples of a multi-core processor include two, four, eight, or greater numbers of processing cores. Alternatively, or in addition, the processor 1603 may include one or more microprocessors configured in tandem via the bus 1601 to enable independent execution of instructions, pipelining, and multithreading. The processor 1603 may also be accompanied with one or more specialized components to perform certain processing functions and tasks such as one or more digital signal processors (DSP) 1607, or one or more application-specific integrated circuits (ASIC) 1609. A DSP 1607 typically is configured to process real-world signals (e.g., sound) in real time independently of the processor 1603. Similarly, an ASIC 1609 can be configured to performed specialized functions not easily performed by a general purposed processor. As a non-limiting example, an ASIC 1609 may be a communications specific circuit capable of sending and receiving information wirelessly (e.g. Wi-Fi, cellular, Bluetooth, GPS). In another non-limiting example, the ASIC 1609 may be a sensor capable of measuring an environmental condition or a physical property (e.g. barometric pressure, temperature, acceleration, inertia). Other specialized components to aid in performing the inventive functions described herein include one or more field programmable gate arrays (FPGA) (not shown), one or more controllers (not shown), or one or more other special-purpose computer chips.

The processor 1603 and accompanying components have connectivity to the memory 1605 via the bus 1601. The memory 1605 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1605 also stores the data associated with or generated by the execution of one or more steps of the methods described herein.

FIG. 17 is a diagram of exemplary components of a mobile terminal 1700 (e.g., cell phone handset) for communications, which is capable of performing the method of FIG. 11, according to one embodiment. In some embodiments, mobile terminal 1701, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory(ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 1703, a Digital Signal Processor (DSP) 1705, and a receiver/transmitter unit including a microphone gain control unit and a speaker gain control unit. A main display unit 1707 provides a display to the user in support of various applications and mobile terminal functions that perform or support the steps as described herein. The display 1707 includes display circuitry configured to display at least a portion of a user interface of the mobile terminal (e.g., mobile telephone). Additionally, the display 1707 and display circuitry are configured to facilitate user control of at least some functions of the mobile terminal. An audio function circuitry 1709 includes a microphone 1711 and microphone amplifier that amplifies the speech signal output from the microphone 1711. The amplified speech signal output from the microphone 1711 is fed to a coder/decoder (CODEC) 1713.

A radio section 1715 amplifies power and converts frequency in order to communicate with a base station, which is included in a mobile communication system, via antenna 1717. The power amplifier (PA) 1719 and the transmitter/modulation circuitry are operationally responsive to the MCU 1703, with an output from the PA 1719 coupled to the duplexer 1721 or circulator or antenna switch, as known in the art. The PA 1719 also couples to a battery interface and power control unit 1720.

In use, a user of mobile terminal 1701 speaks into the microphone 1711 and his or her voice along with any detected background noise is converted into an analog voltage. The analog voltage is then converted into a digital signal through the Analog to Digital Converter (ADC) 1723. The control unit 1703 routes the digital signal into the DSP 1705 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (Wi-Fi), satellite, and the like, or any combination thereof.

The encoded signals are then routed to an equalizer 1725 for compensation of any frequency-dependent impairments that occur during transmission though the air such as phase and amplitude distortion. After equalizing the bit stream, the modulator 1727 combines the signal with a RF signal generated in the RF interface 1729. The modulator 1727 generates a sine wave by way of frequency or phase modulation. In order to prepare the signal for transmission, an up-converter 1731 combines the sine wave output from the modulator 1727 with another sine wave generated by a synthesizer 1733 to achieve the desired frequency of transmission. The signal is then sent through a PA 1719 to increase the signal to an appropriate power level. In practical systems, the PA 1719 acts as a variable gain amplifier whose gain is controlled by the DSP 1705 from information received from a network base station. The signal is then filtered within the duplexer 1721 and optionally sent to an antenna coupler 1735 to match impedances to provide maximum power transfer. Finally, the signal is transmitted via antenna 1717 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 1701 are received via antenna 1717 and immediately amplified by a low noise amplifier (LNA) 1737. A down-converter 1739 lowers the carrier frequency while the demodulator 1741 strips away the RF leaving only a digital bit stream. The signal then goes through the equalizer 1725 and is processed by the DSP 1705. A Digital to Analog Converter (DAC) 1743 converts the signal and the resulting output is transmitted to the user through the speaker 1745, all under control of a Main Control Unit (MCU) 1703 which can be implemented as a Central Processing Unit (CPU) (not shown).

The MCU 1703 receives various signals including input signals from the keyboard 1747. The keyboard 1747 and/or the MCU 1703 in combination with other user input components (e.g., the microphone 1711) comprise a user interface circuitry for managing user input. The MCU 1703 runs a user interface software to facilitate user control of at least some functions of the mobile terminal 1701 as described herein. The MCU 1703 also delivers a display command and a switch command to the display 1707 and to the speech output switching controller, respectively. Further, the MCU 1703 exchanges information with the DSP 1705 and can access an optionally incorporated SIM card 1749 and a memory 1751. In addition, the MCU 1703 executes various control functions required of the terminal. The DSP 1705 may, depending upon the implementation, perform any of a variety of conventional digital processing functions on the voice signals. Additionally, DSP 1705 determines the background noise level of the local environment from the signals detected by microphone 1711 and sets the gain of microphone 1711 to a level selected to compensate for the natural tendency of the user of the mobile terminal 1701.

The CODEC 1713 includes the ADC 1723 and DAC 1743. The memory 1751 stores various data including call incoming tone data and is capable of storing other data including music data received via, e.g., the global Internet. The software module could reside in RAM memory, flash memory, registers, or any other form of writable storage medium known in the art. The memory device 1751 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 1749 carries, for instance, important information, such as the cellular phone number, the carrier supplying service, subscription details, and security information. The SIM card 1749 serves primarily to identify the mobile terminal 1701 on a radio network. The card 1749 also contains a memory for storing a personal telephone number registry, text messages, and user specific mobile terminal settings.

In some embodiments, the mobile terminal 1701 includes a digital camera comprising an array of optical detectors, such as charge coupled device (CCD) array 1765. The output of the array is image data that is transferred to the MCU for further processing or storage in the memory 1751 or both. In the illustrated embodiment, the light impinges on the optical array through a lens 1763, such as a pin-hole lens or a material lens made of an optical grade glass or plastic material. In the illustrated embodiment, the mobile terminal 1701 includes a light source 1761, such as a LED to illuminate a subject for capture by the optical array, e.g., CCD 1765. The light source is powered by the battery interface and power control module 1720 and controlled by the MCU 1703 based on instructions stored or loaded into the MCU 1703.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article. As used herein, unless otherwise clear from the context, a value is “about” another value if it is within a factor of two (twice or half) of the other value. While example ranges are given, unless otherwise clear from the context, any contained ranges are also intended in various embodiments. Thus, a range from 0 to 10 includes the range 1 to 4 in some embodiments.

### 4. REFERENCES

1. Stewart D E, Kucheryavaya A Y, Klassen D K, Turgeon N A, Formica R N, Aeder M I. Changes in Deceased Donor Kidney Transplantation One Year After KAS Implementation. Am J Transplant 2016;16:1834-47.
2. Wissa A, Calogero J, Wereley N, Hubbard J E, Jr., Frecker M. Analytical model and stability analysis of the leading edge spar of a passively morphing ornithopter wing. Bioinspir Biomim 2015;10:065003.
3. Paley D A, Majidi C, Tytell E, Wereley N. Bioinspired roft robotics: preface to the special issue. Bioinspir Biomim 2016;11:020401.
4. Stewart D E, Kucheryavaya A Y, Klassen D K, Turgeon N A, Formica R N, Aeder M I. Changes in Deceased Donor Kidney Transplantation One Year After KAS Implementation. Am J Transplant 2016;16:1834-47.
5. Formica R N, Aeder M, Boyle G, et al. Simultaneous Liver-Kidney Allocation Policy: A Proposal to Optimize Appropriate Utilization of Scarce Resources. Am J Transplant 2016;16:758-66.
6. Koizumi N, DasGupta D, Patel A V, et al. Geographic Variation in Cold Ischemia Time: Kidney vs. Liver Transplantation in the United States, 2003-2011. Transplant Direct 2015;1:e27.
7. Siedlecki A, Irish W, Brennan D C. Delayed graft function in the kidney transplant. Am J Transplant 2011;11:2279-96.
8. Park H S, Hong Y A, Kim H G, et al. Delayed graft function in living-donor renal transplantation: 10-year experience. Transplant Proc 2012;44:43-6.
9. Snyder R A, Moore D R, Moore D E. More donors or more delayed graft function? A cost-effectiveness analysis of DCD kidney transplantation. Clin Transplant 2013;27:289-96.
10. Osband A J, James N T, Segev D L. Extraction Time of Kidneys From Deceased Donors and Impact on Outcomes. Am J Transplant 2016;16:700-3.
11. Lynch R J, Mathur A K, Hundley J C, et al. Improving organ procurement practices in Michigan. Am J Transplant 2009;9:2416-23.
12. Barth R N, Phelan M W, Goldschen L, et al. Single-port donor nephrectomy provides improved patient satisfaction and equivalent outcomes. Ann Surg 2013;257:527-33.
13. Farney A C, Cho E, Schweitzer E J, et al. Simultaneous cadaver pancreas living-donor kidney transplantation: a new approach for the type 1 diabetic uremic patient. Ann Surg 2000;232:696-703.
14. Schweitzer E J, Wilson J, Jacobs S, et al. Increased rates of donation with laparoscopic donor nephrectomy. Ann Surg 2000;232:392-400.
15. Scalea J R, Mezrich J D. Transplant Interrupted: Breaking Routine With Destiny. Ann Surg 2016.
16. Scalea J R, Redfield R R, Rizzari M D, et al. When Do DCD Donors Die?: Outcomes and Implications of DCD at a High-volume, Single-center OPO in the United States. Ann Surg 2016;263:211-6.
17. Scalea J R, Redfield R R, Foley D P. Liver transplant outcomes using ideal donation after circulatory death livers are superior to using older donation after brain death donor livers. Liver Transpl 2016;22:1197-204.

1. A sleeve for an anatomical organ comprising a fabric that is porous with respect to an aqueous solution and has sufficient tensile strength to hold a weight of the sleeve and a weight of a target anatomical organ, wherein the sleeve is shaped to approximate a shape of the target anatomical organ with a first opening configured for inserting the target anatomical organ into the sleeve.

2. The sleeve as recited in claim 1, wherein the fabric is readily cut with surgical shears and the sleeve is further shaped with a different second opening configured to pass a blood vessel for the target anatomical organ.

3. The sleeve as recited in claim 1, further comprising a temperature sensor attached to the fabric of the sleeve.

4. The sleeve as recited in claim 1, further comprising a vibration sensor attached to the fabric of the sleeve.

5. The sleeve as recited in claim 1, wherein the sleeve is shaped to snugly fit the target anatomical organ.

6. The sleeve as recited in claim 1, wherein the fabric includes neoprene.

7. The sleeve as recited in claim 1, wherein the target anatomical organ is one of a group comprising a human kidney, a human heart, a human lung, a human spleen, a human pancreas, and a human eye.

8. A system comprising:
a sleeve for an anatomical organ comprising a fabric that has enough tensile strength to hold a weight of the sleeve and a weight of a target anatomical organ, wherein the sleeve has a first opening configured for inserting the target anatomical organ into the sleeve;
a container configured to hold an aqueous solution;
a temperature sensor configured to be in thermal contact with the sleeve when the sleeve is inside the container, wherein the temperature sensor is configured to produce a plurality of temperature measurements at a corresponding plurality of different temperature times; and
a wireless communication device configured to be in communication with the temperature sensor and configured to wirelessly transmit first data based on the plurality of temperature measurements.

9. The system as recited in claim 8, wherein the sleeve is shaped to approximate a shape of the target anatomical organ.

10. The system as recited in claim 9, wherein the fabric is readily cut with surgical shears and the sleeve is further shaped with a different second opening configured to pass a blood vessel for the target anatomical organ.

11. The system as recited in claim 9, wherein the sleeve is shaped to snugly fit the target anatomical organ.

12. The system as recited in claim 8, wherein the fabric is neoprene.

13. The sleeve as recited in claim 8, wherein the target anatomical organ is one of a group comprising a human kidney, a human heart, a human lung, a human spleen, a human pancreas, and a human eye.

14. The system as recited in claim 8, wherein the temperature sensor is attached to the fabric of the sleeve.

15. The system as recited in claim 14, wherein the temperature sensor is attached to the container.

16. The system as recited in claim 8, further comprising a vibration sensor in mechanical contact with the sleeve when the sleeve is inside the container and configured to produce a plurality of vibration measurements at a corresponding plurality of different vibration times, wherein the wireless communication device is further configured to be in communication with the vibration sensor and configured to wireles sly transmit second data based on the plurality of vibration measurements.

17. The system as recited in claim 16, wherein the vibration sensor is attached to the fabric of the sleeve.

18. The system as recited in claim 16, wherein the vibration sensor is attached to the container.

19. The system as recited in claim 8, further comprising a global positioning system receiver configured to produce a plurality of position measurements at a corresponding plurality of different position times, wherein the wireless communication device is further configured to be in communication with the global positioning system receiver and configured to wirelessly transmit second data based on the plurality of position measurements.

20. The system as recited in claim 19, wherein the global positioning system receiver is attached to the container.

21. The system as recited in claim 8, further comprising a barometric pressure sensor configured to produce a plurality of barometric pressure measurements at a corresponding plurality of different barometric pressure times, wherein the wireless communication device is further configured to be in communication with the barometric pressure sensor and configured to wirelessly transmit second data based on the plurality of barometric pressure measurements.

22. The system as recited in claim 19, wherein the altitude sensor is attached to the container.

23. The system as recited in claim 8, further comprising:
at least one processor; and at least one memory including one or more sequences of instructions, the at least one memory and the one or more sequences of instructions configured to, with the at least one processor, cause the system to perform at least the following
receive the plurality of temperature measurements and determine the first data,
storing the first data in the at least one memory, and
causing the wireless communication device to transmit the first data.

24. The system as recited in claim 8, wherein the wireless communication device is a radio transceiver.

25. An apparatus comprising:
a radio transceiver;
at least one processor; and
at least one memory including one or more sequences of instructions, the at least one memory and the one or more sequences of instructions configured to, with the at least one processor, cause the system to perform at least the following
receive metadata that indicates an anatomical organ;
receive from the radio transceiver first data based on a plurality of temperature measurements at a corresponding plurality of different temperature times from a temperature sensor in thermal contact with the anatomical organ inside a container configured to hold an aqueous solution;
store in the at least one memory the first data in association with the metadata for the anatomical organ;
determine output temperature data based on the first data and output metadata based on the metadata; and
present the output metadata and the output temperature data on a display device.

26. The apparatus as recited in claim 25, the at least one memory and the one or more sequences of instructions further configured to, with the at least one processor, cause the system to perform at least the following:
receive from the radio transceiver second data based on a plurality of position measurements at a corresponding plurality of different position times from a global positioning receiver in contact with a container configured to hold an aqueous solution and the anatomical organ;
store in the at least one memory the second data in association with the metadata for the anatomical organ;
determine output position data based on the second data; and
present the output position data on a display device.

27. The apparatus as recited in claim 25, the at least one memory and the one or more sequences of instructions further configured to, with the at least one processor, cause the system to perform at least the following:
receive patient data that indicates an electronic medical record for a recipient of the anatomical organ;
store in the at least one memory the patient data in association with the metadata for the anatomical organ;
determine output patient data based on the electronic medical record for the recipient; and
present the output patient data on a display device.
